(19) United States
(12) Patent Application Publication
(10) Pub. No.: US 2026/0207885 A1
(43) Pub. Date: Jul. 23, 2026

(54) CATHETER SYSTEM

(71) Applicant: Vigmed AB, Helsingborg (SE)

(72) Inventor: Joakim Henriksson, Helsingborg (SE)

(21) Appl. No.: 19/157,340

(22) PCT Filed: Feb. 15, 2024
(86) PCT No.: PCT/EP2024/053886

§ 371 Date: Aug. 15, 2025

(30) Foreign Application Priority Data
Feb. 17, 2023 (SE) 2350188-5

(51) Int. Cl. A61M 25/00; A61M 5/14; A61M 39/02

(52) U.S. Cl. CPC A61M 25/0014; A61M 5/1413; A61M 39/0247; A61M 2039/0258; A61M 2039/027

(57) ABSTRACT

A closed intravascular (IV) catheter system may extend in a distal-proximal direction along a central axis. The catheter system may include a catheter hub, a tube connector, and a septum. The catheter hub may include a tubular catheter and a catheter hub cavity. A proximal end of the tubular catheter may be attached to a catheter hub body. The catheter hub cavity may be in fluid communication with a lumen of the tubular catheter. The tube connector may be in fluid communication with the catheter hub cavity. The tube connector may extend laterally from the catheter hub body. The septum may be arranged inside the catheter hub cavity proximally to the tube connector. The septum may include a socket between a distal end and a proximal end of the septum. The socket may extend distally past a waist arranged on an internal surface of the catheter hub cavity.

### CROSS-REFERENCE TO RELATED APPLICATIONS

This application claims priority to International Patent Application No. PCT/EP 2024/053886, filed on Feb. 15, 2024, and Swedish Patent Application No. SE 2350188-5, filed on Feb. 17, 2023, the contents of both of which are hereby incorporated by reference in their entirety.

### TECHNICAL FIELD

The present invention relates to a closed IV catheter system. Further, the invention relates to a method for assembling said closed IV catheter system.

### BACKGROUND

Catheters, particularly intravascular (IV) catheters, are used to administer fluids and/or drugs to patients regularly. Fluids are supplied through the catheter through a laterally extending tube, coupled to the catheter through an extension tube coupling present and extending from the catheter hub body.

To protect caretakers and patients from infections caused by exchange of body fluids, such as blood, upon withdrawal of the needle through the catheter, a needle septum may be provided inside the catheter to prevent leakage of blood or other body fluids. The septum also provides a barrier such that no fluids and/or drugs may leak from the catheter hub.

However, when arranging the septum proximally of the laterally extending tube air bubbles may form and aggregate in the area between a distal end surface of the septum and the tube. Such air bubbles may then mix with fluids administered through the tube to a patient, which is unwanted.

Hence, there is an apparent need for an improved catheter instrument.

### SUMMARY

Consequently, the present invention seeks to mitigate, alleviate, eliminate or circumvent one or more of the above identified deficiencies in the art and disadvantages singly or in any combination by providing, according to a first aspect, a closed IV catheter system extending in a distal-proximal direction along a central axis. The catheter system comprises a catheter hub comprising: a tubular catheter attached to a catheter hub body at its proximal end, and a catheter hub cavity in fluid communication with a lumen of the tubular catheter. The catheter system further comprises a tube connector in fluid communication with the catheter hub cavity, the tube connector extending laterally from the catheter hub body, and a septum having a distal end and a proximal end. The septum is arranged inside the catheter hub cavity proximally of the tube connector, and the septum comprises a socket between the distal end and the proximal end. The socket extends distally past a waist arranged on an internal surface of the catheter hub cavity. This is advantageous in that air bubbles are prevented from aggregating in an area between a distal end surface of the septum and the internal surface of the catheter hub cavity. Potentially formed air bubbles may flow further into the patient when administering fluids through the tube connector.

In a second aspect there is provided a method for assembling a closed IV catheter system. The method comprises inserting a needle through a septum, said septum having a distal end and a proximal end, and arranging the septum inside a catheter hub cavity proximally of a tube connector. The septum comprises a socket extending between the distal end and the proximal end, and the socket extends distally past a waist arranged on an internal surface of the catheter hub cavity. The needle extends through the catheter hub cavity and the catheter tube. This method is beneficial since it is simple and provides a closed IV catheter system preventing formation of air bubbles in an area between a distal end surface of the septum and the internal surface of the catheter hub cavity.

Further features of the invention and its embodiments are set forth in the appended claims.

### BRIEF DESCRIPTION OF THE DRAWINGS

These and other aspects, features and advantages of which the invention is capable will be apparent and elucidated from the following description of non-limiting embodiments of the present invention, reference being made to the accompanying drawings, in which:

FIG. 1 is an isometric view of a closed IV catheter system according to one embodiment of the present invention;

FIG. 2 shows a perspective view of a longitudinal cross-section of the closed IV catheter system shown in FIG. 1;

FIG. 3a shows the closed IV catheter system of FIG. 2 as seen from above; FIG. 3b shows the closed IV catheter system of FIG. 3a without a septum;

FIG. 4a-4c show a septum to be arranged in the closed IV catheter system according to one embodiment of the present invention; and

FIG. 5 shows an exploded view of the closed IV catheter system according to one embodiment of the present invention.

### DETAILED DESCRIPTION

Embodiments of the present invention will be described in more detail below with reference to the accompanying drawings in order for those skilled in the art to be able to carry out the invention. The invention may, however, be embodied in many different forms and should not be construed as limited to the embodiments set forth herein. Rather, these embodiments are provided so that this disclosure will be thorough and complete, and will fully convey the scope of the invention to those skilled in the art. The embodiments do not limit the invention, but the invention is only limited by the appended patent claims. Furthermore, the terminology used in the detailed description of the particular embodiments illustrated in the accompanying drawings is not intended to be limiting of the invention.

More specifically, the term “proximal” refers to a location or direction of items or parts of items, during normal use of the closed IV catheter system disclosed herein, is closest to the user, i.e. the clinician, and farthest away from the patient receiving the closed IV catheter system.

Similarly, the term “distal” refers to a location or direction of items or parts of items, during normal use of the closed IV catheter system disclosed herein, is closest to the patient and farthest away from the clinician.

The term “laterally” refers to the direction away from a central axis of the closed IV catheter system, such that at least a vector component perpendicular to the central axis of the closed IV catheter system, wherein the needle and catheter of the assembled closed IV catheter system coincides with the central axis of the closed IV catheter system.

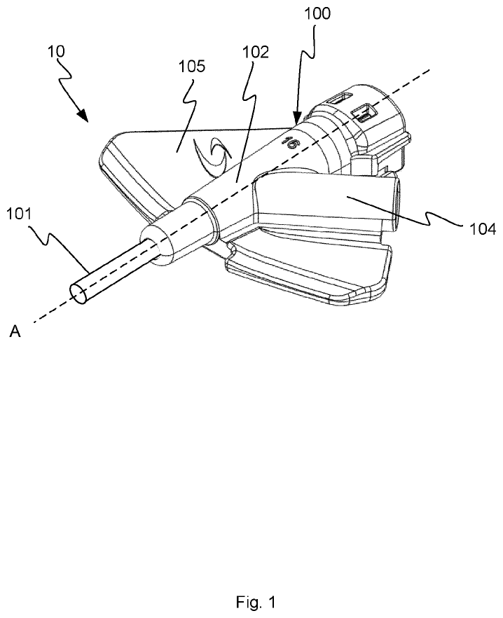

Embodiments of the present invention will now be described below with reference to FIGS. 1 to 5.

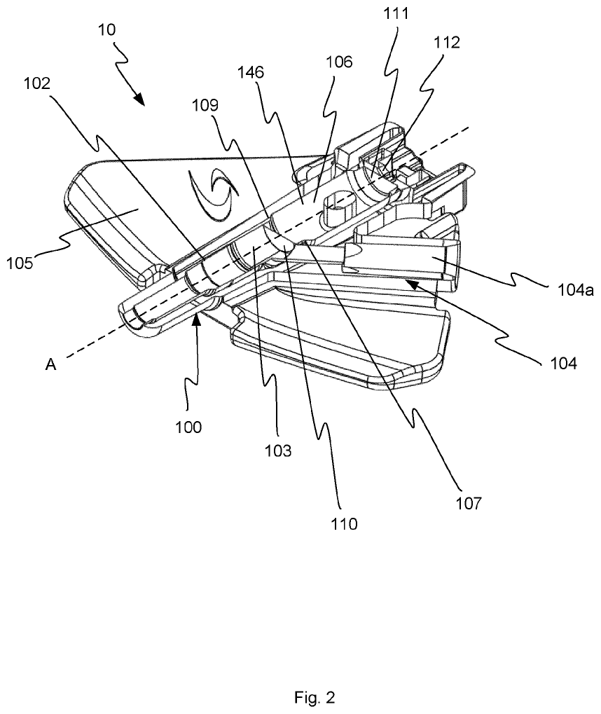

In accordance with FIG. 1, one embodiment of a catheter hub 100 of a closed IV catheter system 10 is illustrated. The closed IV catheter system 10 extends along a central axis A extending in a proximodistal direction. The disclosed catheter system 10 is a closed system, i.e. a system wherein a septum 106 (shown in FIGS. 2, 3a, 4a-c and 5) is provided in a catheter hub body 102, and fluids are withdrawn or introduced through a lateral extension tube (not shown).

As shown in FIG. 1, the catheter hub 100 comprises a longitudinal and tubular catheter 101 at a distal end. The catheter 101 is, in accordance with above, intended to be inserted into a blood vessel of a patient. A proximal end of the catheter 101 is attached to a catheter hub body 102, such that the catheter 101 extends distally from the catheter hub body 102.

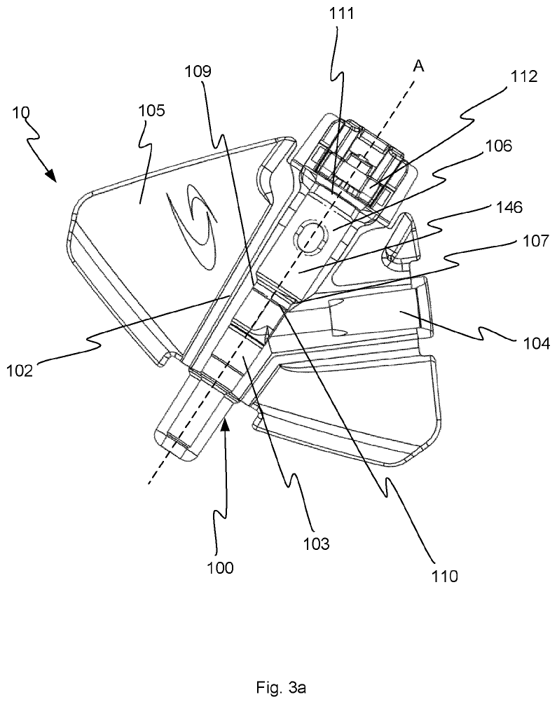

From the catheter hub body 102 a tube connector 104 is provided. The tube connector 104 extends laterally from the catheter hub body 102. The tube connector 104 extends laterally, proximally at an angle from the catheter hub body 102. The tube connector 104 is configured to be connected with a tube (not shown) to allow for infusion from the tube into the tube connector 104, and further into the catheter 101 to the patient. The tube connector 104 has a tube connector proximal end closest to the septum 106 (shown in FIGS. 2 and 3a), and a tube connector distal end closest to the catheter tube 101.

A pair of wings 105 extends laterally from the catheter hub body 102. The wings 105 may have another shape being a substantially flat shape configured to be in contact with a surface of a patient, such as an upper surface of a hand of a patient. The pair of wings 105 is preferably made of an elastic material, such as rubber, silicone or an elastomeric polymer. The tube connector 104 is arranged on top of the wings 105.

Before inserting the catheter 101 into a patient, a needle (not shown) is configured to extend through the septum 106, through the catheter hub cavity 103 and further into the catheter 101 such that a needle tip extends distally from the catheter 101. Once the needle has been inserted intravenously into a patient, the needle is retracted through the closed IV catheter system 10 and the catheter 101 remains intravenously such that fluids, such as a medicament, may be introduced through the tube attached to the tube connector 104.

FIG. 2 shows an isometric view of a cross-section of the catheter system 10, and FIG. 3a shows the cross-section of the catheter system 10 of FIG. 2 as seen from above.

A lumen of the catheter 101 (shown in FIG. 1) is in fluid communication with a catheter hub cavity 103 arranged inside the catheter hub body 102.

The catheter hub body 102 is preferably made through injection moulding, and then of a rigid plastic material suitable for injection moulding and connection and interaction with other parts of the system. Such a suitable material is polycarbonate or a copolymer of polycarbonate and polyester.

The tube connector 104 has a lumen in fluid communication with the hub cavity 103, such that the tube (not shown) may be connected to the tube connector 104 to allow for infusion from the tube into the tube connector 104, further into hub cavity 103 to catheter 101, and finally into the blood stream of the patient. The tube connector 104 may for example be tubular. A suitable material for the tube is polyurethane, polyvinyl chloride, or ethylene vinyl acetate. The tube is configured to be attached to an outer end 104a of the tube connector 104 having a broader width than the remaining portion closer to the axis A.

The septum 106 is arranged in a proximal portion of the catheter hub cavity 103. The septum 106 is arranged proximally of the tube connector 104 and/or the tube. Hence, the catheter hub body 102 comprises the catheter hub cavity 103 and encloses the septum 106. The septum 106 prevents fluids present in the connector tube 104, the catheter 101 or the catheter hub cavity 103 from flowing proximally past the septum 106.

With reference to FIGS. 2, 3a and 3b, an internal surface of the catheter hub cavity 103 comprises a waist 109. The waist 109 may also be referred to as an annular stepped surface 109, or a circumferential step 109. Preferably, the waist 109 is circumferential, i.e. annular.

As shown in FIGS. 2 and 3a, the septum 106 abuts the waist 109. The septum 106 comprises a socket 107 extending distally past the waist 109 arranged on the internal surface of the catheter hub cavity 103. The socket 107 is in contact with the waist 109.

The waist 109 may be curved, as shown in FIGS. 2, 3a and 3b, but may also be shaped as a sharper stepped edge. Hence, the waist 109 may be a pointed edged surface, a chamfered surface or a rounded edged surface. The stepped waist 109 may be circumferential, i.e. annular, as shown in FIGS. 2, 3a and 3b, or may arranged only partly on the internal surface of the distal end of the catheter hub cavity 103.

As shown in FIG. 3a, in an area proximal of the waist 109, the catheter hub cavity 103 has a width being wider than a width of the catheter hub cavity 103 on a distal side of the waist 109. Hence, the waist 109 may be regarded as a result of the transition from the wider part of the catheter hub cavity 103 to a more narrow part of the catheter hub cavity 103.

The waist 109 is thus present at an interface between the wider portion of the catheter hub cavity 103 and more narrow part of the catheter hub cavity 103. The waist 109 prevents a central portion 146 of the septum 106 from being moveable further distally, past the waist 109, inside the catheter hub cavity 103. Hence, the portion of the septum 106 being proximal of the socket 107 cannot extend further distally, past the waist 109, into the catheter hub cavity 103.

FIG. 3b shows the catheter system 10 comprising the catheter hub 100. The catheter hub 100 comprises the wings 105, the catheter hub body 102 comprising the catheter hub cavity 103 and the tube connector 104. In FIG. 3b, the septum 106 is not present in order to more clearly visualize the waist 109 arranged inside the catheter hub cavity 103.

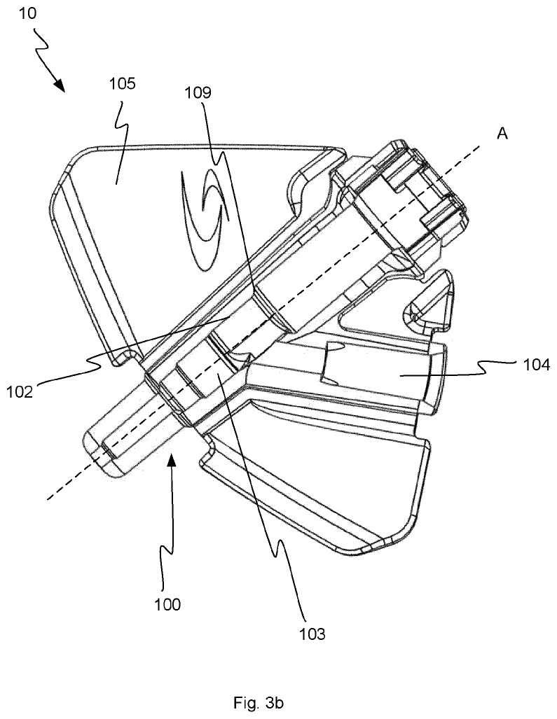

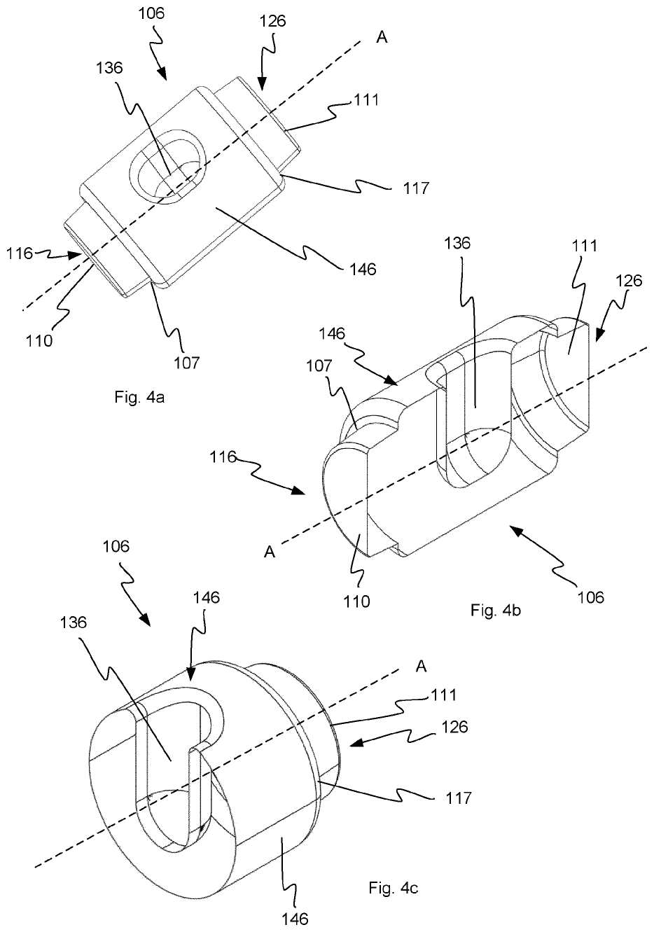

A detailed view of the septum 106 is shown in FIGS. 4a-c. FIG. 4a shows an isometric view of the septum 106. FIGS. 4b and 4c show cross-sectional views of the septum 106 taken along the central axis A and perpendicularly to the axis A, respectively.

The septum 106 is preferably made of an elastic material, such as rubber or silicone. As shown in FIGS. 4a-4c, the septum 106 also extends along the central axis A. The septum 106 has a distal end 116 and a proximal end 126. The septum 106 has a socket 107. The septum 106 has the central portion 146 having a diameter which is wider than a diameter of the distal end 116 and the proximal end 126. As shown in FIGS. 4a-4c, the socket 107 is circumferential. The socket 107 is configured to extend distally past the waist 109 arranged on the internal surface of the catheter hub cavity 103, as shown in FIGS. 2 and 3. Hence, the socket 107 abuts the waist 109 and extends further distally into the catheter hub cavity 103, while the central portion 146 of the septum 106 cannot move or extend past the waist 109.

Further, the septum 106 is provided with a septum cavity 136 arranged in the central portion 146 of the septum 106. The septum cavity 136 extends from an outer surface of the septum central portion 146, into the septum 106 towards and past the central axis A towards an opposite outer surface of the septum 106. Hence, the septum cavity 136 does not extend through the entire septum 106.

In FIGS. 4a-b, each end 116, 126 has a diameter being more narrow than a diameter of the septum central portion 146. Hence, the septum 106 shown in FIGS. 4a-c comprises a proximal socket 117, identical to the socket 107. Therefore, it is arbitrary which end 116, 126 of the septum 106 that is inserted into the catheter hub body 102 first. This makes the assembly of the catheter system 10 easy. In addition, having a socket 107, 117 provided on each side of the septum 106 facilitates the manufacturing of the septum 106, which may be manufactured by injection moulding.

The distal end 116 has a distal end surface 110, and the proximal end 126 has a proximal end surface 111. Alternatively, the septum 106 comprises one socket 107 only and the central portion 146 extends all the way to the proximal end surface 111, and only the distal end 116 has a diameter more narrow than the central portion 146 (not shown).

The socket 107 is configured to contact the waist 109 of the catheter hub cavity 103. More specifically, the socket 107 is arranged to abut the waist 109.

Preferably, as shown in FIG. 3a, the socket 107 is shaped to match the waist 109, such that the shape of the circumferential socket 107 corresponds to the protruding shape of the waist 109. Hence, the socket 107 and the waist 109 matingly contact one another inside the catheter hub body 102. In other words, the circumferential step 109 is shaped to match the outline of the socket 107, such that the socket 107 and the circumferential step 109 may come into direct contact with each other. As shown in FIG. 3a, an angle of the socket 107 corresponds to an angle of the waist 109.

As shown in FIG. 3a, the socket 107 abuts the waist 109, such that the socket 107 of the septum 106 extends distally beyond the waist 109. The socket 107 of the septum 106 extends towards the catheter 101. Further, the distal end surface 110 of the septum 106 is substantially flush, i.e. in level along the central axis A, with the tube connector proximal end. The distal end surface 110 is perpendicular in relation to the longitudinal axis A.

The arrangement of the distal end surface 110 of the septum 106 substantially flush with the tube connector 104 as shown in FIG. 3a prevents the formation of air bubbles and voids between the septum distal end surface 110 and the tube connector 104. This in turn prevents air bubbles formed in the area between the septum distal end surface 110 and the tube connector 104 to flow further through the catheter hub cavity 103 and into the catheter 101, and being administered to a patient.

Preferably, the distal end surface 110 of the septum 106 extends as close to an interface between the proximal end of the tube connector 104 and the catheter hub cavity 103 as possible. Preferably, the distal end surface 110 is flush, i.e. in level along the central axis A, with an internal wall of the proximal end of the tube connector 104.

In one embodiment (not shown), the distal end surface 110 of the septum 106 is angled. Such a septum 106 has a socket 107 as shown in FIGS. 2 and 3, and is provided with a distal end surface 110 being angled relative the longitudinal axis A. The angled distal end surface 110 may be angled such that an angle between the longitudinal central axis A and the distal end surface 110 is between 30 and 50 degrees, such as 45 degrees. Hence, the distal end 116 of the septum 106 is shaped as a truncated cylinder, such that the distal end surface 110 has an elliptic surface area. Preferably, the angled distal end surface 110 is flush with the connector tube 104 at the lateral side adjacent to the connector tube 104, and extends distally into to the catheter hub cavity 103.

The angle of the distal end surface 110 may be the same angle as an angle between the connector tube 104 and the catheter hub body 102. Further preferably, the angled distal end surface 110 may start at the proximal end of the connector tube 104 and extend distally at an angle past the connector tube 104. An angled distal end surface 110 further reduces the risk of having air being trapped or aggregated in the area between the catheter tube 104 and the septum 106 of the catheter hub cavity 103.

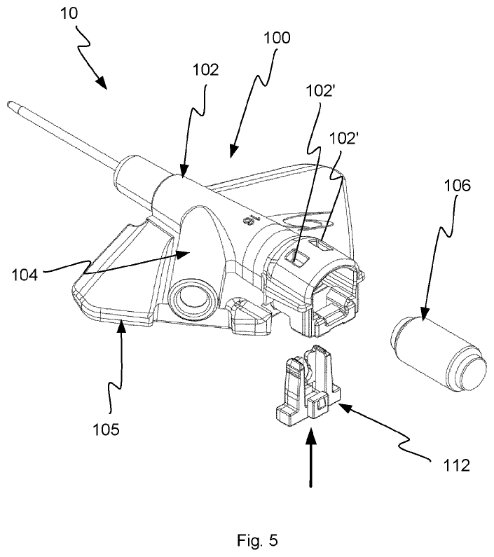

As shown in FIGS. 2 and 3a, the waist 109 prevents the septum 106 from moving distally further into the catheter hub cavity 103, past the waist 109. The waist 109 arranged adjacent to the tube connector 104, proximally of the tube connector 104. A removably attached high pressure clip 112 is arranged proximally of the proximal end surface 111 of the septum 106. The high pressure clip 112 may be a high pressure clip 112 as disclosed in the Applicant's patent application WO2022/010396. The high pressure clip 112 is further described with reference to FIG. 5 below. The high pressure clip 112 prevents the septum 106 from moving proximally, and holds the septum 106 in place in the catheter hub cavity 103 together with the waist 109.

FIG. 5 shows an exploded view of the catheter system 10. FIG. 5 shows the wings 105, the catheter hub 100 comprising the catheter hub body 102 and the tube connector 104, the septum 106, and the high pressure clip 112.

The high pressure clip 112 is shown in its resting state in FIG. 5. The high pressure clip 112 is substantially U-shaped and is made of a slightly flexible material such that upwardly extending legs of the U-shaped clip 112 may flex inwards towards the centre of the U-shaped clip 112. The pressure clip legs each has a flange, which together lock the septum 106 and hold it in its position distal of the high pressure clip 112.

Hence, arranging the high pressure clip 112 in the catheter hub 100 has several functions. Firstly, it locks the needle septum 106 inside the catheter hub 100. In addition, arranging the high pressure clip 112 in the catheter hub 100 improves the assembling process of the catheter instrument 10 to become simpler. The assembly of the catheter system 10 will be more detailed explained with in the following.

Preferably, the high pressure clip 112 is made of a polymeric material (such as a plastic) or a metal material.

To assemble the catheter system 10, a needle (not shown) is inserted through the septum 106 to form a subassembly, which is then inserted into the catheter hub body 102. When inserting the subassembly comprising the needle and the septum 106 into the catheter hub cavity 103, the subassembly is pushed distally into the catheter hub body 102 until the socket 107 abuts the waist 109. Hence, the waist 109 blocks the septum 106 from moving further distally. The needle (not shown) will extend through the catheter hub cavity 103, and into and through the catheter tube 101 (shown in FIGS. 1 and 5). The insertion of the needle will push the socket 107 of the septum 106 slightly distally of the opening from the connector tube 104. However, upon withdrawal of the needle during use, the septum 106 will slide proximally to the position as shown in FIGS. 2 and 3a.

Then, the high pressure clip 112 is inserted into the catheter hub 100 from below as indicated by the bold arrow in FIG. 5. The upwardly extending legs of the high pressure clip 112 will thus engage upper apertures 102′ of the catheter hub body 102, and hold the septum 106 in place.

According to another embodiment, to assemble the catheter system 10, the septum 106 is inserted into the catheter hub body 102. When inserting the septum 106 into the catheter hub cavity 103, the socket 107 will abut the waist 109. Hence, the waist 109 blocks the septum 106.

Further, the high pressure clip 112 is inserted into the catheter hub 100 from below as indicated by the bold arrow in FIG. 5. The upwardly extending legs of the high pressure clip 112 will thus engage upper apertures 102′ of the catheter hub body 102, and hold the septum 106 in place.

A needle (not shown) is then inserted into the catheter system 10, along the axis A, through the septum 106 and through the catheter hub cavity 103, and into and through the catheter tube 101 (shown in FIG. 1). The insertion of the needle will push the distal end 116 of the septum 106 slightly distally of the opening from the connector tube 104. However, upon withdrawal of the needle during use, the septum 106 will slide proximally to the position as shown in FIGS. 2 and 3a.

Alternatively, the needle is introduced before the high pressure clip 112 is inserted into the catheter hub 100.

Also, the broadest diameter of the septum 106 in its resting state, i.e. as shown in FIGS. 4a-4c is slightly wider than when the septum 106 is arranged inside the catheter hub body 102. Due to its elasticity, the septum 106 will become somewhat elongated when pushed into the catheter hub cavity 103 compared to its length in its resting state outside the catheter system 10. Hence, a width of the catheter hub cavity 103 is slightly smaller than the diameter of the septum 106 in its resting state. This also facilitates a stable arrangement of the septum 106 inside the catheter hub cavity 103.

When the needle has been withdrawn from the catheter hub 100, the septum 106 will become sealed, such that the hub cavity 103 is marked off from the surroundings in the proximal direction. For this reason, the septum 106 is preferably of a suitable rubber material or silicone.

Preferably, the catheter hub body 102 is made from a polymeric material, such as a plastic material, being more rigid than an elastic material forming the wings 105. Preferably, the catheter hub 100 and the wings 105 are formed integrally, from different materials. During manufacturing, the catheter hub body 102 is formed through injection moulding of a first material, preferably a hard plastic material. Once the catheter hub body 102 is moulded, the injection moulding tool is turned and the wings 105 are injection moulded on top of the catheter hub body 102 in a flexible material. The wings 105 and catheter hub body 102 are formed from any one of the materials as described above.

In the claims, the term “comprises/comprising” does not exclude the presence of other elements or steps. Furthermore, although individually listed, a plurality of means, elements or method steps may be implemented by e.g. a single unit or processor. Additionally, although individual features may be included in different claims, these may possibly advantageously be combined, and the inclusion in different claims does not imply that a combination of features is not feasible and/or advantageous. In addition, singular references do not exclude a plurality. The terms “a”, “an”, “first”, “second” etc. do not preclude a plurality. Reference signs in the claims are provided merely as a clarifying example and shall not be construed as limiting the scope of the claims in any way.

1. A closed intravascular (IV) catheter system extending in a distal-proximal direction along a central axis, the catheter system comprising:
a catheter hub including:
a tubular catheter attached to a catheter hub body at a proximal end of the tubular catheter; and
a catheter hub cavity in fluid communication with a lumen of the tubular catheter;
a tube connector in fluid communication with the catheter hub cavity, the tube connector extending laterally from the catheter hub body; and
a septum having a distal end and a proximal end, the septum arranged inside the catheter hub cavity proximally to the tube connector
wherein the septum includes a socket between the distal end and the proximal end; and
wherein the socket extends distally past a waist arranged on an internal surface of the catheter hub cavity.

2. The closed IV catheter system according to claim 1, wherein the socket is in contact with the waist.

3. The closed IV catheter system according to claim 2, wherein a shape of the socket matches a corresponding shape of the waist.

4. The closed IV catheter system according to claim 1, wherein the waist is arranged proximally to the tube connector.

5. The closed IV catheter system according to claim 1, wherein a distal end surface of the septum is flush with a proximal end of the tube connector.

6. The closed IV catheter system according to claim 1, wherein a distal end surface of the septum is angled relative the central axis.

7. The closed IV catheter system according to claim 1, wherein:
the waist is configured as an interface between a proximal portion of the catheter hub cavity and a distal portion of the catheter hub cavity; and
the proximal portion of the catheter hub cavity is wider than the distal portion of the catheter hub cavity.

8. The closed IV catheter system according to claim 1, wherein a diameter of the socket is larger than a diameter of the distal end of the septum.

9. The closed IV catheter system according to claim 1, wherein the waist is annular.

10. The closed IV catheter system according to claim 1, wherein the waist is a rounded waist.

11. The closed IV catheter system according to claim 1, wherein the septum includes a further socket arranged at the proximal end of the septum.

12. The closed IV catheter system according to claim 1, wherein the septum further includes a septum cavity extending laterally from an outer surface of the septum, towards and past the central axis.

13. The closed IV catheter system according to claim 1, wherein the septum is composed of an elastomer.

14. The closed IV catheter system according to claim 1, wherein the septum is formed in one piece.

15. A method for assembling a closed intravascular (IV) catheter system according to claim 1, the method comprising:
inserting a needle through a septum, the septum having a distal end and a proximal end; and
arranging the septum inside a catheter hub cavity proximally of a tube connector;
wherein the septum includes a socket extending between the distal end and the proximal end;
wherein the socket extends distally past a waist arranged on an internal surface of the catheter hub cavity; and
wherein the needle extends through the catheter hub cavity and the catheter tube

16. The method according to claim 15, wherein arranging the septum inside the catheter hub cavity includes the needle moving the septum distally past a proximal end of the tube connector.

17. The method according to claim 16, further comprising inserting a high pressure clip into the catheter hub proximally of the septum, such that the high pressure clip prevents the septum from moving proximally past the high pressure clip.

18. The method according to claim 15, further comprising inserting a high pressure clip into the catheter hub, proximally of the septum, such that the high pressure clip prevents the septum from moving proximally past the high pressure clip.

19. The closed IV catheter system according to claim 1, wherein the waist is a chamfered waist.

20. The closed IV catheter system according to claim 13, wherein the elastomer is silicone or rubber.
